(19) United States
(12) Patent Application Publication
(10) Pub. No.: US 2021/0315087 A1
(43) Pub. Date: Oct. 7, 2021

(54) ELECTROSTATIC DISCHARGE BRUSHES

(71) Applicants: Da-Peng LI, Shanghai (CN); Chee-Kheong CHOOI, Shanghai (CN); Zhi-Yuan ZHAO, Shanghai (CN); Hewlett-Packard Development Company, L.P., Spring, TX (US)

(72) Inventors: Da Peng Li, Shanghai (CN); Chee-Kheong Chooi, Shanghai (CN); Zhi Yuan Zhao, Shanghai (CN)

(21) Appl. No.: 17/250,628

(22) PCT Filed: Nov. 21, 2018
(86) PCT No.: PCT/CN2018/116665

§ 371 Date: Feb. 12, 2021

(51) Int. Cl. H05F 3/00

(52) U.S. Cl. CPC H05F 3/00

(57) ABSTRACT

Electrostatic discharge (ESD) brushes are described. An ESD brush includes a base (102) and a plurality of bristles(104-1, 104-2, 104-3, . . . , 104-n, 304-1, 304-2) integrated with the base(102).

### BACKGROUND

Electronic devices, such as printing devices, rely on usage of static electricity for their operation. For example, laser printing devices use a laser beam to scan an area of a sheet to develop a pattern of electrostatic charge. The electrostatic charge attracts a powdered ink onto the sheet, which is further bonded to the sheet. Once printed, the electrostatic charge accumulated on the sheet is removed.

### BRIEF DESCRIPTION OF THE DRAWINGS

FIG. 1 illustrates an electrostatic discharge (ESD) brush, in accordance with an example of the present subject matter;

FIGS. 2(a) and 2(b) illustrate an ESD brush, in accordance with another example of the present subject matter;

FIG. 3 illustrates an ESD brush, in accordance with another example of the present subject matter;

FIG. 4 illustrates different stages of manufacturing of an ESD brush, in accordance with an example implementation of the present subject matter;

FIG. 5 illustrates a printing device, in accordance with an example implementation of present subject matter; and

FIG. 6 illustrates a method of manufacturing an ESD brush, in accordance with an example implementation of present subject matter.

### DETAILED DESCRIPTION

In electronic devices, such as printing devices, an electrostatic charge may develop during operation. For example, in document feeders, an electrostatic charge may develop on multiple sheets stored in the document feeder due to friction between the sheets and friction between the sheets and parts of the printing device. Accumulation of electrostatic charge on sheets may result in sticking of multiple sheets leading to multi-feeding, attraction of airborne contaminants to the sheets causing printing voids, damaging of scanning sensor causing scanning and copying errors, and sometimes ink dispersion in printing devices' cartridges.

Electrostatic discharge (ESD) brushes are used to avoid development of electrostatic charge. An ESD brush is made of two components, a base and bristles. The ESD brush collects the electrostatic charge accumulated on various surfaces, such as sheets, through the bristles, and allows the electrostatic charge to flow to the ground via the base of the ESD brush.

The ESD brush manufacturing process may involve grouping the bristles to form different sets, followed by attaching the grouped bristles to the base of the ESD brush. The manufacturing of the ESD brush involves multiple process steps, including but not limited to, cleaning, grouping, adhesion, and pasting.

Since, the process steps involved in the manufacturing of the ESD brush have various stages and utilize different components, the overall manufacturing process is time consuming, tedious and complicated. Further, since the ESD brush is produced by combining various components, regular functioning of the ESD brush causes an early wear-tear of the ESD brush.

The present subject matter describes durable ESD brushes along with techniques of manufacturing the durable ESD brushes. In an example implementation of the present subject matter, the techniques of manufacturing include manufacturing an ESD brush through a quick and efficient process. The manufactured ESD brush includes an integrated base and bristles which makes the ESD brush durable and less prone to wear-and-tear.

In an example, the manufacturing of the ESD brush includes punching a sheet of a first material to form a base of the ESD brush along with the bristles. The punching of the sheet of the first material carves out a base and the bristles, integrated with each other, thereby forming the ESD brush. The manufacturing of the ESD brush further includes pressing the bristles to provide a predefined angle to the bristles, with respect to the base of the ESD brush. The pressing of the bristles aligns the bristles at the predefined angle with respect to the base of the ESD brush.

Thus, the manufacturing of ESD brushes based on the techniques described herein eliminates various steps of cleaning, grouping, adhesion, and pasting, which are otherwise involved in the production of ESD devices, thereby providing an efficient and effective production of ESD brushes. Further, since the ESD brush is formed from a single sheet of the first material, the manufacturing process is simple and the ESD brush thus formed is also durable.

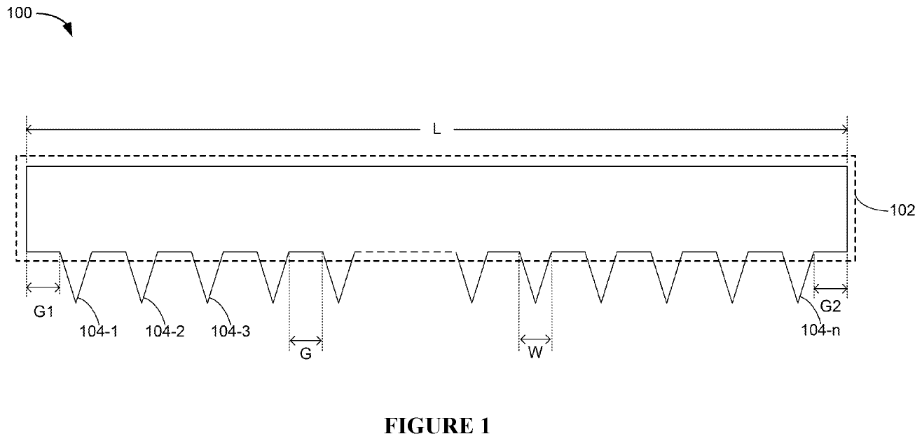

The above techniques are further described with reference to FIG. 1 to FIG. 6. It should be noted that the description and the figures merely illustrate the principles of the present subject matter along with examples described herein, and should not be construed as a limitation to the present subject matter. It is, thus understood that various arrangements may be devised that although not explicitly described or shown herein, embody the principles of the present subject matter. Moreover, all statements herein reciting principles, aspects, and implementations of the present subject matter, as well as specific examples thereof, are intended to encompass equivalents thereof.

FIG. 1 illustrates an ESD brush 100, in accordance with an example implementation of present subject matter. The ESD brush 100 includes a base 102 and multiple bristles 104-1, 104-2, 104-3, . . . , 104-n formed integrally with the base 102. For the ease of reference, the bristles 104-1, 104-2, 104-3, . . . , 104-n have been referred to as bristles 104.

Further, the base 102 and the multiple of bristles 104 may be made of a first material. For example, the first material may include any non-metallic conductor, including but not limited to, Polyethylene terephthalate (PET), conductive polycarbonate, and conductive poly crystalline silicon. In an example, the base 102 may have a length proportional to the size of a surface from which the electrostatic charge is to be collected. For instance, the length ‘L’ of the base 102 may be about 8.3 inches corresponding to an A4 size sheet. In another example, the length of the base 102 may be about 11.7 inches, corresponding to an A3 size sheet.

The bristles 104 may have different shapes, including but not limited to, triangular, oval, circular, and square. However, for the sake of simplicity, the bristles 104 have been shown in the triangular shape. Further, each of the multiple bristles 104 may have a width W of less than 2 millimetres (mm), where the width W is the width of the bristles 104 at the point of contact of the base 102 and the bristles 104. The width W of less than 2 mm, of the each of the bristles 104, may reduce the friction between ESD brush 100 and other components, such as a print sheet, during operation. Furthermore, the ESD brush 100 may have a predefined gap G between each pair of adjacent bristles. For example, the predefined gap G may be at least 4 mm. The predefined gap G may reduce the friction between the ESD brush 100 and other components, such as a print sheet, during operation.

In an example, the ESD brush 100 may be mounted onto a device, including but not limited to, a scanner, a printer, and other devices for operation. Accordingly, the ESD brush 100 may have a predefined gap G1 at a first end and a predefined gap G2 at a second end. The predefined gaps G1 and G2 may allow the ESD brush 100 to be mounted on a device without being in contact with other parts of the device. In an example, the predefined gap G1 and G2 may be at least 8 mm. Further, in case of wear-and-tear of the ESD brush 100, changing the ESD brush is easier due to mount ability of the ESD brush 100. In another example, the ESD brush 100 may be fixed on the device using an adhesive. The process involving pasting the ESD brush 100 on the device avoids a mounting process, making the assembly of the device simple and convenient.

In an example, the ESD brush 100 may be electrically connected to a ground terminal (not shown) of the device, such that the electrostatic charge collected by the ESD brush 100 is transferred to the ground via the ground terminal. In operation, when a component, such as a printing sheet surface comes in contact with the ESD brush 100, the bristles 104 may collect the electrostatic charge accumulated on the surface of the printing sheet and may pass them to the ground, through the ground terminal. It would be noted that the ground terminal may be electrically connected to the ground/earth electrical connection of the device.

In an example, the ESD brush 100 may be mounted in a printing device, such as, a printer, to collect and avoid development of electrostatic charge on printing sheets. The ESD brush 100 may further be connected to a ground terminal of the printer. In operation, the bristles 104 may collect charge from the printing sheet during the operation of the printer. The electrostatic charge collected by the bristles of the ESD brush 100 may be transferred to the ground terminal of the printer.

In an example, the ESD brush 100 may be mounted in a space between a document feeder and a printer cartridge of the printer. The installation of the ESD brush 100 between the document feeder and a printer cartridge may allow collection of electrostatic charge accumulated on the printing sheet, before the printing sheet is fed into the printer cartridge for printing operation. Similarly, another ESD brush 100 may also be mounted between the print cartridge/print drum and a document excreter to remove any electrostatic charge accumulated during the printing. It would be noted that while use of two ESD brushes 100 has been described, a printer may use a single ESD brush 100 as well, either between the document feeder and the printer cartridge, or between the print cartridge/print drum and the document excreter. Further, it would be noted that the ESD brush 100 may also be placed at different positions, other than described above.

In yet another example, the ESD brush 100 may be mounted in a scanner, such as a sheet-fed scanner. The bristles 104 may collect charge from the sheets being scanned and transfer the accumulated charge to the ground terminal. On a scanner, in an example, the ESD brush 100 may be mounted in a space between an automatic document feeder and the scanning elements, such as projector lamps and cameras. The installation of the ESD device at such position may allow collection of electrostatic charge accumulated on the sheets being scanned.

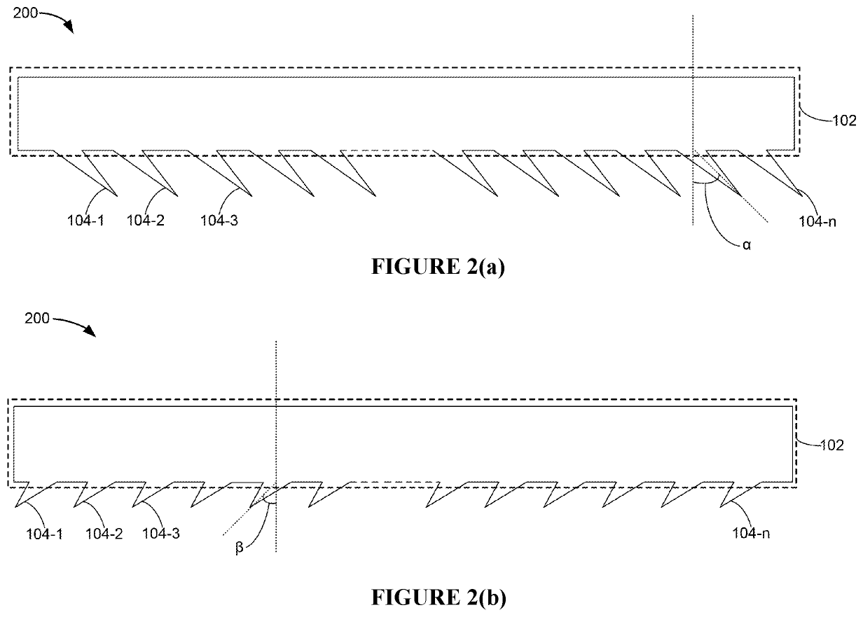

FIG. 2(a) illustrates an ESD brush 200, in accordance with an example implementation of present subject matter. As described earlier, the ESD brush 200 may include a base 102 and the bristles 104-1, 104-2, 104-3, . . . , 104-n, hereinafter referred to as bristles 104, formed on the base, where the bristles 104 are formed integrally with the base 102. In an example, the bristles 104 may be folded on a first side of the ESD brush 200. Further, the base 102 and the bristles 104 may be aligned at an angle α with respect to each other. That is, the plane of the surface of the base 102, and the plane of the surface of the bristles 104 are at the angle α. In an example, the angle α between the base 102 and the bristles 104 may be an acute angle.

FIG. 2(b) illustrates an ESD brush 200, in accordance with another example implementation of present subject matter. The ESD brush 200 includes a base 102 and multiple bristles 104-1, 104-2, 104-3, . . . , 104-n, hereinafter referred to as bristles 104, formed integrally on the base 102. In an example, the bristles 104 may be folded on a second side of the ESD brush 200. The base 102 and the bristles 104 may be aligned at an angle β. That is, the plane of the surface of the base 102, and the plane of the surface of the bristles 104 is at the angle β. In an example, the angle β between the base 102 and the bristles 104 may be an acute angle.

In an example, the angle between the base 102 and the bristles 104 may be varied based on implementation of the ESD brush 100 into the device. For example, if the ESD brush 100 is implemented in a printer, the angle between the base 102 and the bristles 104 may be based on the capacity of a document feeder. That is, the angle between the base 102 and the bristles 104 may be varied based on a level of stacking of sheets in the document feeder. In an example, the angle between the base 102 and the bristles 104 may be varied to ensure that there is a predefined gap between the bristles 104 and the sheets in the document feeder. In an example, the predefined gap may be around 2 mm.

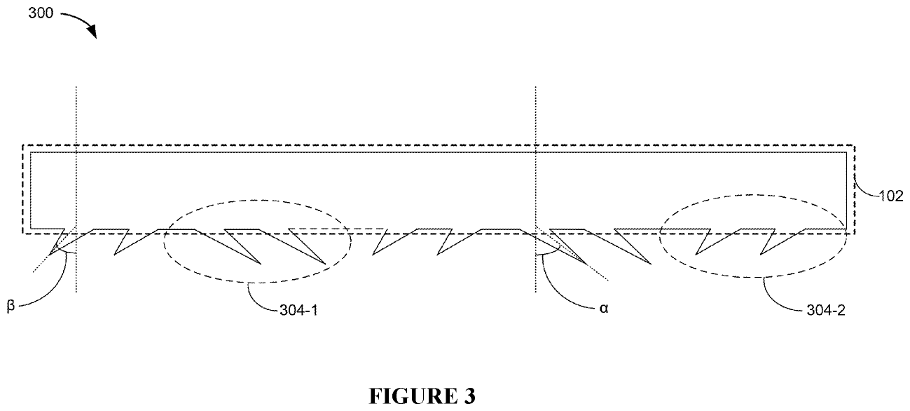

FIG. 3 illustrates an ESD brush 300, in accordance with an example implementation of present subject matter. The ESD brush 100 includes a base 102 and multiple bristles 304-1 and 304-2, hereinafter referred to as bristles 304, formed on the base 102, where the bristles 304 are integrally formed integrally with the base 102.

In an example, a set of bristles 304-1 are aligned at a first predefined angle α with respect to the base 102. Further, another set of bristles 304-2 are aligned at a second predefined angle β with respect to the base 102. In an example, the first predefined angle α and the second predefined angle β are acute angles.

In an example, the set of bristles 304-1 may be folded to incline on a first side of the ESD brush 300. Further, the set of bristles 304-2 may be folded to incline on a second side of the ESD brush 300. Such an arrangement of the bristles 304 may reduce the area of contact of the ESD brush at a specific region on the sheet. Thus, the effective friction at a specific part of the sheet may be distributed enough by the ESD brush 300, which may render the effect of friction negligible on the discharging process.

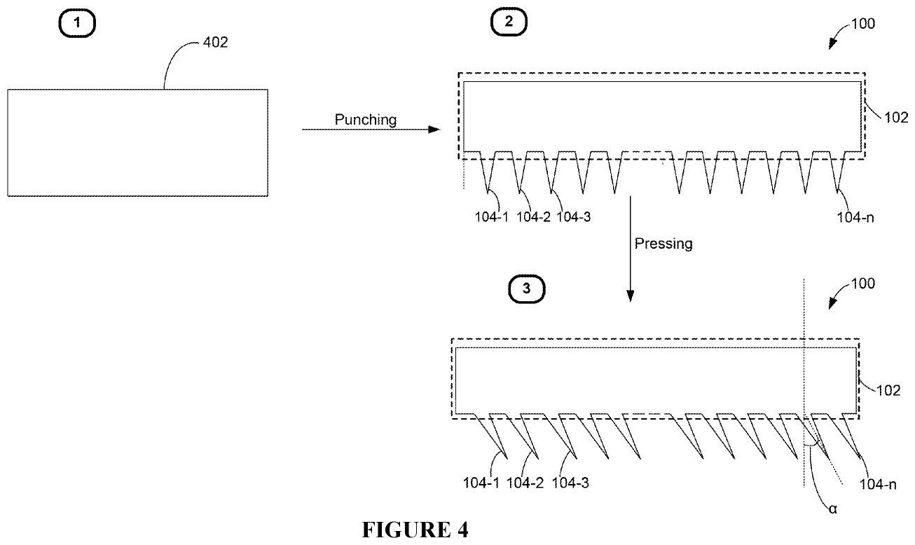

FIG. 4 illustrates various stages of manufacturing of an ESD brush, such as an ESD brush 100, in accordance with an example implementation of the present subject matter. The manufacturing the ESD brush 100 may be divided into one or more stages, including but not limited to, stage 1, 2, and 3. The three stages involved in the manufacturing of the ESD brush 100 is not intended to be construed as a limitation, and the manufacturing of the ESD brush may be divided into any number of stages

The manufacturing of the ESD brush 100 starts at stage 1. At stage 1, a sheet 402 of a first material is taken. The first material may be a non-metallic conductor, including but not limited to, Polyethylene terephthalate (PET), conductive polycarbonate, and conductive poly crystalline silicon. In an example, the sheet 402 may have different sizes. The size of the sheet 402 may be decided based on the size of the ESD brush 100. For instance, to manufacture an ESD brush 100 to collect the electrostatic charge off the surface of an A4 size printing sheet, an ESD brush 100 having length of about 8.3 inches may be used. To manufacture the ESD brush 100 having length more than 8.3 inches, the sheet 402 may have a length of at least 8.3 inches. Further, the width of the sheet may be decided based on the overall width of the ESD brush 100.

Further, at stage 2 a portion of the sheet 402 of the first material is punched out to obtain an ESD brush 100. The ESD brush 100 so obtained may include a base 102 and multiple bristles 104-1, 104-2, 104-3, . . . , 104-n, hereinafter referred to as bristles 104, formed integrally with each other.

In an example of the present subject matter, the sheet of the first material may be punched through a die of a predefined shape. The predefined shape may include an integrated contour of the base 102 and the bristles 104. The bristles 104 may collect the charge accumulated on the sheet and may transfer the same to the base 102. The base 102 may further transmit the electrostatic charge to the ground via a grounding terminal.

At stage 3, the ESD brush 100 is further pressed to provide a predefined angle α to the bristles 104 with respect to the base 102. In an example, the predefined angle α may allow the ESD brush 100 to be used for different levels of sheet stackings in a document feeder of a printing device.

Therefore, the ESD brush 100 manufactured based on the techniques described above eliminates various steps of cleaning, grouping, adhesion, and pasting, generally involved in the production of ESD brush, thereby providing an efficient and effective production of ESD brushes. Further, since the ESD brush is formed from a single sheet of first material, the manufacturing process is simple and the ESD brush thus formed is also durable.

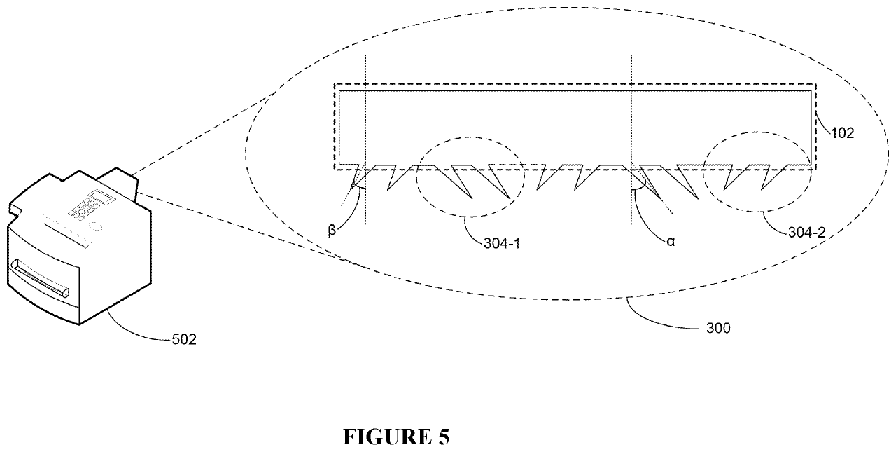

FIG. 5 illustrates a printing device 502, in accordance with an example of present subject matter. The printing device 502 may comprise an ESD brush 300 having a base 102 and bristles 304-1 and 304-2, hereinafter referred to as bristles 304. The bristles 304 may comprise a first set of bristles 304-1 integrally formed on the base 102 and inclined at a predefined angle α with respect to the base 102. Further, the bristles 304 may also include a second set of bristles 304-2 integrally formed on the base 102, inclined at a predefined angle β with respect to the base 102.

In an example, the printing device 502 may have the ESD brush 300 installed between a space between a document feeder (not shown) and a cartridge (not shown). The installation of the ESD brush 300 between the document feeder and the cartridge may allow to collect the electrostatic charge accumulated on the surface of the sheets before the sheets are fed into the cartridge for printing.

In an example, the ESD brush 300 may be mounted inside the printing device. The mounting may be done on support structures (not shown) formed on the printing device 502 to support ESD brush 300. In another example, the ESD brush 300 may be pasted inside the printing device 502 via an adhesive.

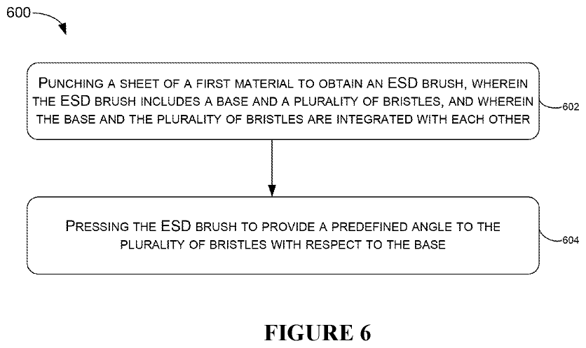

FIG. 6 illustrates a method of manufacturing an ESD brush, such as an ESD brush 100, in accordance to an example implementation of the present subject matter. The order in which the method 500 is described is not intended to be construed as a limitation, and any number of the described method blocks may be combined in any order to implement the method 600, or any alternative methods. Furthermore, the method 500 may be implemented by electronic circuits, or processor(s) through any suitable hardware, or combination thereof for manufacturing the ESD brush 100.

At block 602, a sheet of a first material is punched to obtain an ESD brush, where the ESD brush includes a base and multiple bristles, and where the base and the bristles are integrated with each other. In an example, a sheet of the first material may be punched to obtain an ESD brush, such as an ESD brush 100, where the ESD brush 100 includes a base 102 and multiple bristles 104 integrated with each other. In the example, the sheet of the first material may be punched using a die of a predefined shape, where the predefined die may be an integrated contour of the base 102 and the bristles 104.

At block 604, the ESD brush is pressed to provide a predefined angle to the bristles with respect to the base. In an example, the ESD brush, such as the ESD brush 100, may be pressed to provide a predefined angle to the bristles 104 through a die.

Although implementations of the present subject matter have been described in language specific to methods and/or structural features, it is to be understood that the present subject matter is not limited to the specific methods or features described. Rather, the methods and specific features are disclosed and explained as example implementations of the present subject matter.

1. A method of manufacturing an electrostatic discharge (ESD) brush, the method comprising:
punching a sheet of a first material to obtain an ESD brush, wherein the ESD brush includes a base and a plurality of bristles, and wherein the base and the plurality of bristles are integrated with each other; and
pressing the ESD brush to provide a predefined angle to the plurality of bristles with respect to the base.

2. The method as claimed in claim 1, wherein the predefined angle between the base and the plurality of bristles is an acute angle.

3. The method as claimed in claim 1, wherein the punching the sheet of the first material is based on a die of a predefined shape, and wherein the predefined shape is an integrated contour of the base and the plurality of bristles.

4. The method as claimed in claim 1, wherein the pressing comprises folding the plurality of bristles with respect to the base, through a die.

5. The method as claimed in claim 1, wherein the plurality of bristles includes a first set of bristles at a first predefined angle with respect to the base, and wherein the plurality of bristles includes a second set of bristles at a second predefined angle with respect to the base.

6. The method as claimed in claim 5, wherein the first set of bristles is inclined towards one side of the base and the second set of bristles is inclined towards another side of the base.

7. An ESD brush comprising:
a base; and
a plurality of bristles formed on the base, wherein the plurality of bristles is formed integrally with the base.

8. The ESD brush as claimed in claim 7, wherein the base and the plurality of bristles are made of a first material.

9. The ESD brush as claimed in claim 8, wherein the first material is one of Polyethylene terephthalate (PET), conductive polycarbonate, and conductive poly crystalline silicon.

10. The ESD brush as claimed in claim 8, wherein the first material is a non-metallic conductor.

11. The ESD brush as claimed in claim 7, wherein the base and the plurality of bristles are aligned at an acute angle.

12. The ESD brush as claimed in claim 11, wherein the plurality of bristles includes a first set of bristles aligned at a first predefined angle and the plurality of bristles includes a second set of bristles aligned at a second predefined angle.

13. The ESD brush as claimed in claim 7, wherein each bristle of the plurality of bristles has a width less than 2 mm.

14. The ESD brush as claimed in claim 7, wherein two consecutive bristles of the plurality of bristles have a distance of at least 4 mm therebetween.

15. A printing device comprising an ESD brush, wherein the ESD brush comprises:
a base;
a first set of bristles integrally formed on the base, wherein the first set of bristles is inclined at a first predefined angle with respect to the base; and
a second set of bristles integrally formed on the base, wherein the second set of bristles is inclined at a second predefined angle with respect to the base.
